(19) United States
(12) Patent Application Publication
(10) Pub. No.: US 2010/0247427 A1
(43) Pub. Date: Sep. 30, 2010

(54) Methods of inhibiting cell growth and methods of enhancing radiation responses

(75) Inventors: Javier F. Torres-Roca, St. Petersburg, FL (US); Douglas P. Calvin, Tampa, FL (US); Madhavi Sekharam, Tampa, FL (US); Hua E. Yu, Glendora, CA (US); Richard Jove, Glendora, CA (US)

Correspondence Address: MICHAEL BEST & FRIEDRICH LLP, 100 East Wisconsin Avenue, Suite 3300, Milwaukee, WI, 53202, US

(73) Assignee: University of South Florida, Tampa, FL (US)

(21) Appl. No.: 11/581,293

(22) Filed: Oct. 16, 2006

Related U.S. Application Data
(60) Provisional application No. 60/726,909, filed on Oct. 14, 2005.

(51) Int. Cl. C12N 13/00; A61K 31/282; A61K 51/00; A61P 35/04; A61N 5/00

(52) U.S. Cl. 424/1.11; 435/173.1; 514/492; 604/20

(57) ABSTRACT

Provided herein are methods of enhancing the radiation response of a cell expressing activated Stat1, Stat3, or Stat5. Methods for synergistically affecting a cell expressing activated Stat1, Stat3, or Stat5 are also described. The described methods may also be used to synergistically affect or enhance the radiation response of a cell in a subject.

### CROSS-REFERENCE TO RELATED APPLICATIONS

This application claims priority to U.S. Provisional Application No. 60/726,909 filed Oct. 14, 2005 which is incorporated herein by reference.

### BACKGROUND

Signal transducer and activator of transcription (Stat)-family proteins are latent cytoplasmic transcription factors that convey signals from the cell surface to the nucleus on activation by cytokines and growth factors. See Yu and Jove, Nat Rev 4:97-105 (2004) and Levy and Darnell, Nat Rev Mol Cell Biol 3:651-662 (2002). Engagement of cell surface receptors by polypeptide ligands, such as interleukin-6 (IL-6) or epidermal growth factor, induces tyrosine phosphorylation of Stat proteins by Janus kinase, growth factor receptor tyrosine kinases, and Src family tyrosine kinases. The phosphorylated Stat protein in the activated dimeric form then translocates to the nucleus and affects expression of genes having Stat-binding sites in their promoters. Under normal physiologic conditions, activation of Stat proteins is rapid, transient and regulates expression of genes that control fundamental biological processes, including cell proliferation, survival, and development.

Numerous studies have detected active Stats, particularly Stat1, Stat3 and Stat5, in diverse human tumor specimens, including myeloma, leukemia, lymphoma, melanoma and carcinomas from prostate, ovary and head and neck. Stat activity is established as essential for malignant transformation of cultured cells by many oncogenic signaling pathways. For example, the Src, Janus kinase, and epidermal growth factor receptor family tyrosine kinases are frequently activated in breast cancer cells and induce Stat3 activation. Blocking tyrosine kinase pathways with selective pharmacologic inhibitors results in decreased Stat3 activity, growth inhibition, and apoptosis. Activation of Stat3 and Stat5 in tumor cells has been shown to affect expression of genes involved in controlling cell cycle progression, apoptosis, and angiogenesis.

Platinum complexes are pharmacologic inhibitors capable of inhibiting the function and/or downstream effects of Stat activation. See U.S. Patent Application Publication No. 2005/0074502 which is incorporated herein by reference in its entirety. Cells having little or no activated Stat were unaffected by treatment with the platinum complexes. In contrast, cells having activated Stat3, such as breast cancer cells, demonstrated growth inhibition and decreased viability after treatment with the platinum complexes.

### SUMMARY OF THE INVENTION

In one aspect, methods of enhancing the radiation response of a cell having activated Stat1, Stat3, or Stat5 are provided. The cell is contacted with an effective amount of a platinum complex selected from the group consisting of CPA-1, CPA-3, CPA-7 and pharmaceutically acceptable salts thereof and an effective amount of radiation. The platinum complex enhances the radiation response of the cell.

In another aspect, methods of affecting a cell having activated Stat1, Stat3 or Stat5 are provided. The cell is contacted with synergistically effective combination of CPA-7 or a pharmaceutically acceptable salt of CPA-7 and radiation. Contact with CPA-7 and radiation exerts synergistic effects on the cell.

In yet another aspect, methods of affecting a cell having activated Stat1, Stat3, or Stat5 in a subject are provided. A synergistically effective combination of CPA-7 or a pharmaceutically acceptable salt of CPA-7 and radiation is administered to the subject. The combination exerts synergistic effects on the cell.

In a still further aspect, methods of enhancing the radiation response of a cell having activated Stat1, Stat3, or Stat5 in a subject are provided. An effective amount of a platinum complex selected from the group consisting of CPA-1, CPA-3, CPA-7 and pharmaceutically acceptable salts thereof and an effective amount of radiation are administered to the subject. Administration of the platinum complex enhances the radiation response of the cell.

### BRIEF DESCRIPTION OF THE DRAWINGS

FIG. 1 is an electrophorectic mobility shift assay (EMSA) demonstrating activated Stat3 in a variety of cancer cell types.

FIG. 2A is a graph of an MTT assay showing cell viability of LnCap cells after treatment with various amounts of CPA-7 for the indicated time intervals.

FIG. 2B is a graph of an MTT assay showing cell viability of DU145 cells after treatment with various amounts of CPA-7 for the indicated time intervals.

FIG. 3 is a graph of a clonogenic assay showing the surviving fraction of DU145 cells after treatment with various combinations of CPA-7 and radiation.

FIG. 4 is a set of FACs analyses demonstrating the percentage of cellular apoptosis induced after pretreatment with the indicated amounts of CPA-7 and radiation.

FIG. 5 is a graph depicting the results of a TUNEL assay showing the percent apoptosis of DU145 cells after contact with CPA-7, Cisplatin and 2 Gy radiation or combinations of either CPA-7 and radiation or Cisplatin and radiation.

FIG. 6 is a graph demonstrated the effect of CPA-7 and radiation on cellular viability as measured by trypan blue assay.

FIG. 7A is a graph showing the effect of various dosages of CPA-7 in combination with various amounts of radiation on cellular viability of LnCap cells as measured by MTT assay.

FIG. 7B is a graph showing the effect of various dosages of CPA-7 in combination with various amounts of radiation on cellular viability of DU145 cells as measured by MTT assay.

FIG. 8 is a photograph of a Western blot for Survivin, Mcl-1, Bcl-X_{L} and actin after treatment of DU145 cells with the indicated amounts of CPA-7 and radiation for 24 or 48 hours.

### DETAILED DESCRIPTION

Provided herein are methods of enhancing the radiation response or inhibiting the growth of a cell having activated Stat1, Stat3, or Stat5. The inventors found that certain platinum complexes block the effects of Stat activation and enhance the radiation responsiveness in cells having activated Stat1, Stat3, or Stat5. Additionally, the combination of a platinum complex and radiation may have a synergistic cellular effect. Cells may be contacted in vitro, in vivo (i.e., in a subject) or ex vivo. Suitable subjects include, but are not limited to, mammals, such as humans.

The enhancement of the radiation response may lower the effective amount of radiation required to exert cellular effects or it may increase the radiation responsiveness of the cell and result in increased cellular effects of the radiation treatment. Cellular effects may include, but are not limited to, one or more of increased apoptosis of cells, increased cell death, increased inhibition of cell growth, reduced tumor volume, reduced tumor burden, clearance of a tumor, inhibition of tumor growth or tumor cell proliferation, inhibition of metastases, reduced metastases and enhanced survival of a subject bearing tumor or cancer cells expressing activated Stats. “Synergistic cellular effects” indicates that the total cellular effect of the combination of a platinum complex and radiation is greater than the sum of the individual cellular effects of the platinum complex alone and radiation alone.

An effective amount of the platinum complex is an amount sufficient to enhance responsiveness to radiation. Suitably, an effective amount of radiation is used in combination with the platinum complex. One of skill in the art will understand that the effective amount of the platinum complex and radiation may be inversely related, i.e., if a high dosage of platinum complex is used, it may be combined with a lower dose of radiation to achieve cellular effects. Alternatively, the maximum tolerated dose of the platinum complex, the radiation or both the platinum complex and the radiation may be used to achieve greater cellular effects. Suitably, a synergistically effective combination of the platinum complex and radiation may be used.

A synergistically effective combination of a platinum complex and radiation is a combination that gives a cellular effect, which may be therapeutic, that is greater than the sum of the cellular effects of the platinum complex alone and radiation alone.

Any cell expressing activated Stat1, Stat3 or Stat5 may be utilized in the methods. Suitably the cells are cancer cells, tumor cells or transformed cells. Cancer cells include, but are not limited to, a breast cancer cell, a lung cancer cell, an ovarian cancer cell, a head and neck cancer cell, a melanoma cell, a prostate cancer cell, a multiple myeloma cell, a lymphoma cell, a leukemia cell, a gastric cancer cell, a glioma cancer cell, an ovary cancer cell, a colon cancer cell, and a pancreatic cancer cell. Tumor cells include cells derived from or within any tumor. Tumor refers to any manifestation of a hyperproliferative disorder, including, e.g., a solid tumor mass, a system of tumor nodules and/or cancer of the hematopoietic system. Examples of tumors suitably treated in accordance with the presently described methods include, but are not limited to, carcinomas of the breast, head and neck squamous cell carcinomas, prostate carcinomas, ovarian carcinomas, skin melanomas, leukemias and lymphomas. Transformed cells include any cell which has been altered such that its growth or proliferative capacity is increased relative to a non-transformed cell.

As will be understood, any neoplastic disease characterized by abnormal Stat activation is suitably ameliorated as described herein. The cells may be from a mammal, including but not limited to, human, monkey, chimpanzee, ape, dog, cat, horse, cow, or pig. Activation of Stat1, Stat3, and Stat5 may be determined by any means known to those of skill in the art, including but not limited to, electrophorectic mobility shift assays (EMSA), and Western blots using antibodies specific for the activated form of the protein.

Platinum complexes useful in the methods include, but are not limited to, CPA-1, CPA-3 and CPA-7. These and other platinum complexes are described in U.S. Patent Application Publication Nos. 2005/0074502 and 2005/0080131, each of which is incorporated herein by reference in its entirety. The platinum complexes can be prepared using standard chemical synthesis methods and materials known in the art.

Radiation useful in the methods includes but is not limited to, X-rays, gamma rays, radioactive seeds and radionuclides. Radiation may be administered externally or internally using conventional radiation dosing schedules. For example, radiation therapy may be given daily, 5 days per week. Radiation dosage depends on a number of factors including tumor type, age, weight and condition of the patient, as well as other factors typically considered by the skilled clinician. The typical dose for a solid epithelial tumor may range from 50 to 70 grays (Gy) or more, while lymphomas (white cell) tumors might receive doses closer to 20 to 40 Gy given in daily doses. The total dose can be given in daily fractions using external beam radiation or the total dose can be given via other methods such as implants that deliver radiation continuously over a given timeframe. Depending on the implant type, the dose may be given as a fraction (e.g., High Dose Rate, HDR) over minutes or hours. Alternatively, permanent seeds may be implanted (such as in the prostate) that slowly deliver radiation until the seeds become inactive. It is envisioned that administration of a platinum complex in combination with radiation will result in increased cellular effects for given dose of radiation. Alternatively, lower dosages of radiation may be employed when used in combination with a platinum complex than are typically used in radiation therapy alone.

Platinum complex compositions for use in the described methods also include pharmaceutically acceptable salts of the platinum complexes. Pharmaceutically acceptable salts include salts of the platinum complexes that are prepared with acids or bases, depending on the particular substituents found on the platinum complexes described herein. Examples of a pharmaceutically acceptable base addition salts include, but are not limited to, sodium, potassium, calcium, ammonium, or magnesium salt. Examples of pharmaceutically acceptable acid addition salts include, but are not limited to, hydrochloric, hydrobromic, nitric, phosphoric, carbonic, sulphuric, and organic acids like acetic, propionic, benzoic, succinic, fumaric, mandelic, oxalic, citric, tartaric, and maleic. Pharmaceutically acceptable salts of platinum complexes may be prepared using conventional techniques known to those of skill in the art.

It will be appreciated by those skilled in the art that some of the platinum complexes may contain one or more asymmetrically substituted carbon atoms which can give rise to stereoisomers. All such stereoisomers, including enantiomers, and diastereoisomers and mixtures, including racemic mixtures thereof are included within the scope of the invention.

The platinum complexes of the subject invention are potent and selective disruptors of Stat activity. CPA-1 and CPA-7 have been demonstrated to strongly disrupt Stat3 activity and interfere with its ability to bind to its consensus binding sequence. See Turkson et al., Mol Cancer Ther 3:1533-1542 (2004), which is incorporated herein by reference in its entirety. In addition, CPA-3 strongly disrupts Stat5 activity. These platinum complexes induce cell growth inhibition and apoptosis in cancer-tells, transformed cells and tumor cells with persistently active Stats. Malignant cells with aberrant or constitutive Stat signaling are highly sensitive to these platinum complexes.

General cytotoxicity of the platinum complexes to normal cells is minimal. Because CPA-1 and CPA-7 selectively block the growth and replication of cells that contain activated Stat3, while only slowing the growth of cells having normal Stat3, these platinum complexes are attractive candidates for inhibiting the growth of cells having activated Stats. In addition, strong apoptosis is induced by platinum complexes in malignant cells that harbor activated Stats. As shown in the Examples, apoptois correlates with suppression of aberrant Stat activity in these cells.

The platinum complexes also exhibit anti-tumor activity in melanoma and colon tumors in vivo. Platinum complexes have been shown to down-modulate Stat activation and the expression of Stat regulated anti-apoptotic factors. The platinum complexes may exhibit anti-tumor activity toward tumors having activated Stats due to these cellular affects.

Platinum complexes can be delivered to a cell either through direct contact with the cell or via a carrier. Carriers for delivering compositions to cells are known in the art and include, for example, liposomes. Another method for delivering a platinum complex to a cell comprises attaching the platinum complexes to a protein or nucleic acid that is targeted for delivery to a cell. Published U.S. Patent Application Nos. 2003/0032594 and 2002/0120100 disclose amino acid sequences that can be coupled to another composition and that allow the composition to be translocated across biological membranes. Published U.S. Patent Application No. 2002/0035243 also describes compositions for transporting biological moieties across cell membranes for intracellular delivery. In addition, the platinum complex may be conjugated to an antibody specific for a cell surface marker on a cell. A further suitable method for delivery of the platinum complexes known in the art includes nanoparticle delivery, such as aptamer-conjugated nanoparticle delivery.

Administration of the platinum complexes, pharmaceutically acceptable salts of the platinum complexes or compositions comprising the platinum complexes can be accomplished by any suitable technique. The platinum complexes may be administered by any suitable route including, for example, oral, nasal, rectal, and parenteral routes of administration. As used herein, the term parenteral includes subcutaneous, intradermal, intravenous, intramuscular, intraperitoneal, and intrathecal administration, such as by injection.

Platinum complexes, pharmaceutically acceptable salts of the platinum complexes or compositions comprising the platinum complexes and radiation can be administered continuously or at discrete time intervals as can be readily determined by a person skilled in the art. An ordinarily skilled clinician can determine a suitable amount of a platinum complex and radiation to be administered to a subject. The amount of platinum complex and radiation administered is a therapeutically effective amount, i.e., an amount sufficient to produce a cellular effect. One of skill in the art will understand that the therapeutically effective amount of the platinum complex and radiation may be inversely related, i.e., if a high dosage of platinum complex is used, it may be combined with a lower dose of radiation to achieve therapeutic efficacy. Alternatively, the maximum tolerated dose of the platinum complex, the radiation or both the platinum complex and the radiation may be used to achieve higher therapeutic efficacy. The platinum complex and a radiation may be administered in an amount such that the therapeutic effect is synergistic, i.e. greater the sum of individual therapeutic effects of the platinum complex alone and radiation alone.

The specific therapeutically effective dose level for any particular subject will depend upon a variety of factors, including the disorder being treated and the severity of the disorder; activity of the specific compound employed; the specific composition employed; the age, body weight, general health, sex and diet of the subject; route of administration; the rate of excretion of the platinum complex employed; the duration of the treatment; other pharmaceuticals used in combination or coincidental with the platinum complex and like factors well known in the medical arts. For example, it is well within the level of ordinary skill in the art to start doses at levels lower than those required to achieve the desired therapeutic effect and to gradually increase the dosage until the desired effect is achieved.

If desired, the effective daily dose may be divided into multiple doses for purposes of administration. Consequently, single dose compositions may contain such amounts or submultiples thereof to make up the daily dose. As noted, those of ordinary skill in the art will readily optimize effective doses and co-administration regimens as determined by good medical practice and the clinical condition of the individual patient.

For example, suitable total daily dosages of compositions including CPA-7 may provide about 1 mg/kg to about 10 mg/kg, about 4 mg/kg to about 6 mg/kg, and/or about 5 mg/kg. Doses are suitably administered about twice or about three times per week. Daily administration of lower dosages is also contemplated. Administration is suitably continued until tumor burden is reduced in a subject by at least 50%. Most suitably, administration is continued until the tumor is no longer detected in a patient, i.e., the patient is in complete clinical remission.

The platinum complex is suitably administered concurrent with or prior to administration of the radiation. Suitably the platinum complex is administered from about 10 minutes to about 1 week prior to administration of the radiation. More suitably, the platinum complex is administered from about 1 hour to about 2 days prior to administration of the radiation. Most suitably, the platinum complex is administered from about 4 hours to about 1 day prior to administration of the radiation.

Compositions containing platinum complexes useful in the methods can be formulated according to known methods for preparing pharmaceutically useful compositions. Formulations are described in detail in a number of sources which are well known and readily available to those skilled in the art. For example, Remington's Pharmaceutical Science, by E. W. Martin, describes formulations which can be used in the disclosed methods. In general, the compositions will be formulated such that an effective amount of the platinum complex is combined with a suitable carrier in order to facilitate effective administration of the composition.

The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The form will depend on the intended mode of administration and therapeutic application. The platinum complex compositions also suitably include conventional pharmaceutically acceptable excipients which are known to those skilled in the art. Examples of excipients for use with the platinum complexes include ethanol, dimethyl sulfoxide, glycerol, alumina, starch, and equivalent carriers and diluents. To provide for the administration of such dosages for the desired application, pharmaceutical compositions will comprise between about 0.1% and 99%, and suitably between about 1 and 15% by weight of the total of one or more of the platinum complexes based on the weight of the total composition including the carrier or diluent.

The compositions may be administered utilizing liposome technology, slow release capsules, implantable pumps, and biodegradable containers. These delivery methods may provide a uniform dosage over an extended period of time. The platinum complexes may also be administered in their salt derivative forms or cystalline forms.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

It also is specifically understood that any numerical range recited herein includes all values from the lower value to the upper value, i.e., all possible combinations of numerical values between the lowest value and the highest value enumerated are to be considered to be expressly stated in this application. For example, if a concentration range is stated as 1% to 50%, it is intended that values such as 2% to 40%, 10% to 30%, or 1% to 3%, etc., are expressly enumerated in this specification. If a concentration range is “at least 5%, it is intended that all percentage values up to and including 100% are also expressly enumerated. These are only examples of what is specifically intended.

The following Examples are provided to assist in a further understanding of the invention. The particular materials, methods and conditions employed are intended to be illustrative of the invention and are not limiting upon the scope of the invention.

### EXAMPLES

### Example 1

The following materials and methods were used throughout the rest of the Examples.

Cell culture. Human prostate cancer, DU145 and LnCap cells were obtained from the American Type Culture Collection (ATCC) and were maintained in RPMI (with or without phenol red) medium containing 10% fetal bovine serum in an atmosphere of 95% air and 5% CO₂.

CPA-7 treatment. Prostate cancer or human lung fibroblast cells were plated at required density in 100 mm, 60 mm, 6-well or 96-well plates. After 18 hours of adherence, cells were treated or left untreated with CPA-7 in 50% DMSO for 4 hours or 24 hours, irradiated and incubated for the indicated time intervals. For the pulse labeling experiments, the cells were treated with CPA-7 for 4 hours, irradiated, and the cells were post incubated in drug free medium for the indicated time intervals.

Irradiation procedure. The cells were irradiated by means of Cesium-137 (J. L. Shepherd Mark 1 Model 68a).

Cytotoxicity assays. The drug and/or radiation sensitivity was determined by tetrazolium based colorimetric MTT assay. The MTT dye quantifies metabolically viable cells that reduce the yellow tetrazolium salt to bluish-purple formazan crystals. 5×10⁴ DU145 cells/well and 1.5×10⁵ LNCap cells/well were plated in 96 well plates. After 18 hours of adherence, the cells were treated with 0-5 μM CPA-7 (8 wells per treatment) for 4 hours or 24 hours and were exposed to 0-10 Gy Cesium-137. After 48 hours or 72 hours of further incubation, the medium was replaced with 2 mg/ml MTT solution. Cells were incubated at 37° C. for 3 hours and the resulting formazan crystals were solubilized with DMSO, and absorption was measured at 540 nm using a multiscanner autoreader (Dynatec Mr 5000; Cantilly, Va.)

Clonogenesis assay. Clonogenic survival assays after 2 Gy of radiation were performed as previously described by Gupta et al., Cancer Res 61:4278-82 (2001) which is incorporated herein by reference in its entirety. Plating efficiency (PE) for each cell line was determined, prior to survival fraction at 2 Gy (SF2) determination. Cells were plated so that 50-100 colonies would form per plate and incubated overnight at 37° C. to allow for adherence. Cells were then radiated with 2 Gy using a Cesium Irradiator (J. L. Shepherd, Model I 68A, San Fernando, Calif.). Exposure time was adjusted for decay every three months. After irradiation, cells were incubated for 10-14 days at 37° C. before being stained with crystal violet. Only colonies with at least 50 cells were counted. SF2 was determined by the following formula: SF2=number of colonies/total number of cells plated×plating efficiency.

Cell proliferation and viability. After overnight attachment, DU145 cells were pretreated with 2.5 and 5.0 μM CPA-7 for 4 hours or 24 hours, with corresponding controls treated with DMSO. Cells were irradiated at 0, 2 and 5 Gy and cell numbers were measured by counting using a hemacytometer after an additional 24, 48, and 72 hours. Cell viability was measured by trypan blue exclusion or MTT assay.

Nuclear extract preparation and gel shift assays. DU145 cells were treated for 4 hours or 24 hours with 2.5 and 5.0 μM CPA-7. Corresponding controls were treated with vehicle alone (DMSO). The cells were irradiated and nuclear extracts were prepared 1 hour and 24 hours later for electrophoretic mobility shift assay (EMSA). Nuclear extracts were prepared by high-salt extraction into 30 to 70 μL buffer [20 mmol/L HEPES (pH 7.9), 420 mmol/L NaCl, 1 mmol/L EDTA, 20% glycerol, 20 mmol/L NaF, 1 mmol/L Na₃V0₄, 1 mmol/L Na₄P₂O₇, 1 mmol/L DTT, 0.5 mmol/L phenylmethylsulfonyl fluoride, 0.1 μmol/L aprotinin, 1 μmol/L leupeptin, and 1 μmol/L antipain] as previously described by Yu et al., Science 269:81-83 (1995), which is incorporated herein by reference. For EMSA, 5 μg of total nuclear protein were used for each lane. EMSA was done using a ³²P-labeled oligonucleotide probe containing a high-affinity cis-inducible element (hSIE, m67 variant) derived from the c-fos gene promoter that binds activated Stat3 proteins. Following incubation of radiolabeled probes with nuclear extracts, protein-DNA complexes were resolved by non-denaturing PAGE and detected by autoradiography. Stat3 protein was supershifted in the EMSA by preincubation with Stat3 antibody (C-20×, Santa Cruz Biotechnology, Santa Cruz, Calif.).

Western immunoblotting. For protein analyses, 1×10⁵ logarithmically-growing cells were plated in 100 mm plates. After overnight attachment, the cells were treated with 2.5 and 5.0 μM CPA-7 for 4 hours or 24 hours and irradiated at 0, 2, and 5 Gy. Cell lysates were prepared 24 and 48 hours after irradiation with ice-cold RIPA lysis buffer. The lysates were clarified by centrifugation, and protein concentrations of resultant supernatants were determined by Bio-Rad assay (Bio-Rad, Hercules, Calif.). Equal amounts of proteins were separated by SDS-10% polyacrylamide gel electrophoresis, and were transferred onto nitrocellulose membranes. The membranes were blocked with 5% milk in Tris-buffered saline for 4 hours at room temperature, and were incubated overnight at 4° C. with blocking solution containing the desired antibody. Membranes were washed and incubated with horseradish peroxidase-conjugated secondary antibodies and the activities were detected by chemiluminescence (Amersham) as described. The following antibodies were used for the respective proteins: polyclonal rabbit anti-human Mcl-1, Bcl-X_{S/L}, Stat3, and polyclonal goat anti-human actin were obtained from Santa Cruz (Santa Cruz, Calif.), polyclonal rabbit anti-human phospho-stat3 (Tyr705) was obtained from Cell Signaling Technology (Beverly, Mass.), and polyclonal rabbit anti-human survivin was obtained from Alpha Diagnostic International (San Antonio, Tex.).

TUNEL assay. Cells were labeled for apoptotic DNA strand breaks by terminal deoxyribonucleotidyl transferase-mediated dUTP nick end labeling (TUNEL) reaction using a flow cytometry assay (Roche Applied Science, Indianapolis, Ind.) according to the instructions of the supplier. DU145 cells were treated with CPA-7 for 4 hours, then irradiated, and subjected to the TUNEL assay 24 to 48 hours later. To determine cellular viability, cells were harvested by trypsinization and counted by trypan blue exclusion assay at 24 and 48 hours after irradiation. All experiments were done in triplicate.

### Example 2

### Stat 3 is Activated in Cell Lines Derived from a Variety of Cancers

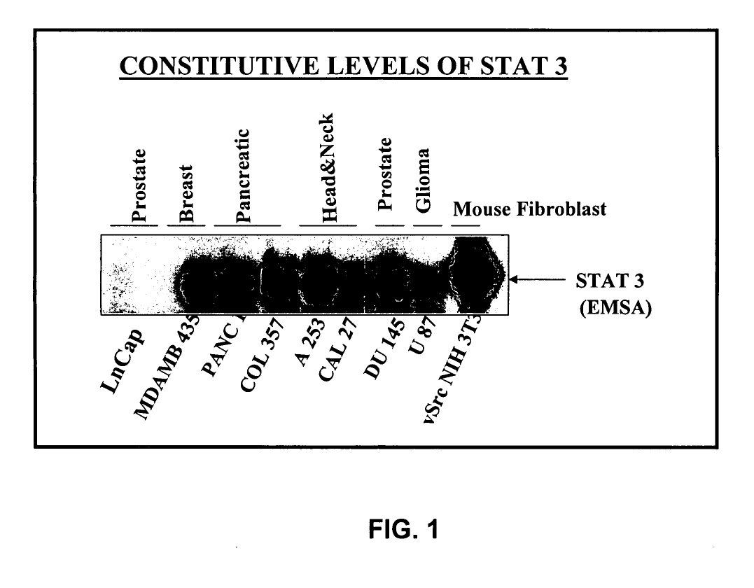

Nuclear extracts were prepared as described above for each of the following cell lines: LnCap (prostate), MDA-MB-435 (breast), PANC-1 (pancreatic), COL 357 (pancreatic), A253 (head and neck), CAL 27 (head and neck), DU 145 (prostate), U87 (Glioma) and vSrc NIH 3T3 cells (mouse fibroblast) control. The tissue source for each cell line is indicated in the parentheses. The results of the EMSA are depicted in FIG. 1 and demonstrate that many cancer-derived cell lines have activated Stat3.

### Example 3

### CPA-7 is Cytotoxic in Cells Expressing Activated Stat3 as Measured by MTT Assay

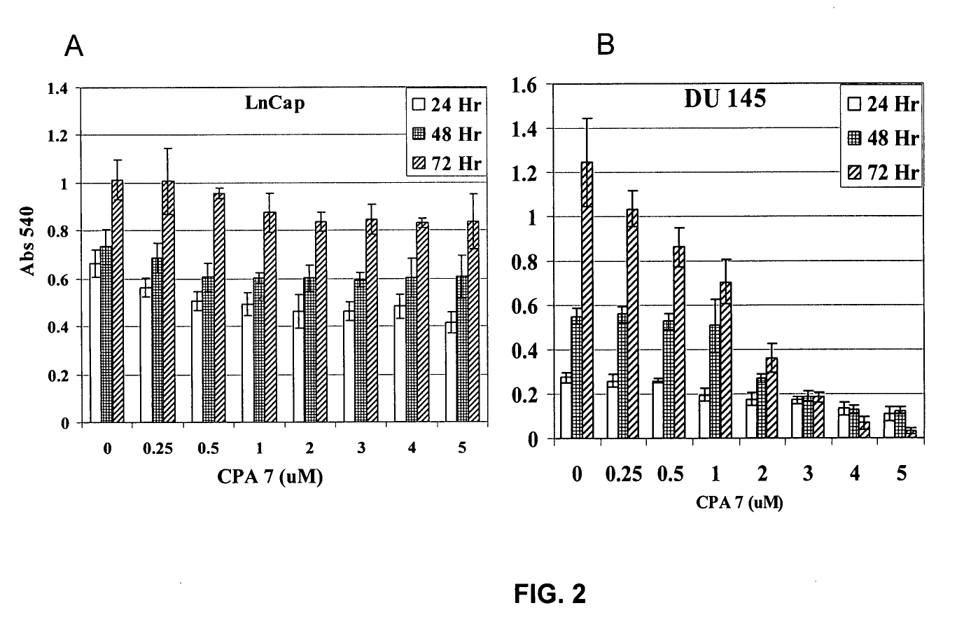

To establish whether CPA-7 was cytotoxic in prostate cancer cell lines and whether any observed cytotoxicity was restricted to cell lines with activated Stat3, DU145 and LnCap cells were treated with the indicated amounts of CPA-7 or left untreated. The cells were incubated for 24, 48 or 72 hours. The cells were washed and an MTT assay was performed to assess the viability of the cells. As shown in FIG. 2B, CPA-7 had a dose-dependent cytotoxic effect on DU145 cells, which have activated Stat3. At low concentrations, CPA-7 inhibited the growth of the DU145 cells and at higher concentrations the CPA-7 was cytotoxic to the cells. In contrast, as shown in FIG. 2A, CPA-7 failed to induce similar cytotoxicity in LnCap prostate cancer cells, which do not have activated Stat3 (See FIG. 1). These results suggest that Stat3 activation may be required for cytotoxicity of CPA-7.

### Example 4

### Clonogenic Assay for Effect of Combination of CPA-7 and Radiation

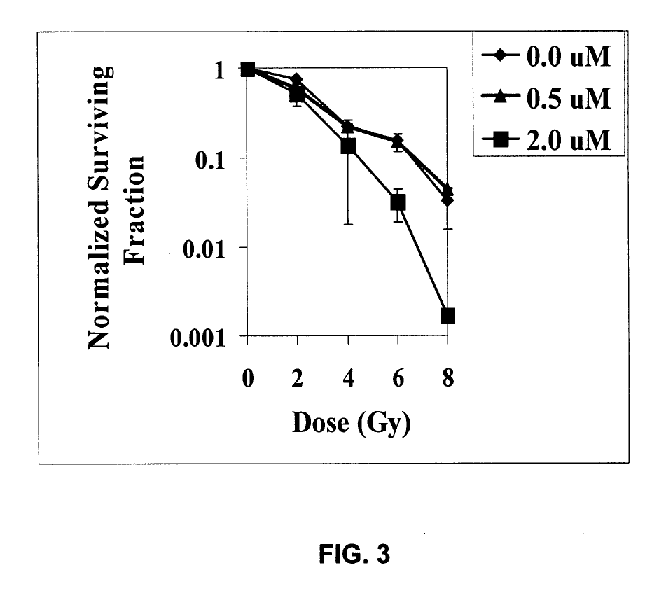

To determine whether CPA-7 would enhance the radiation response, clonogenic assays of DU145 cells were performed following treatment with CPA-7 and radiation therapy. In FIG. 3, the cells were pretreated with CPA-7 for four hours prior to administering radiation. CPA-7 enhanced the radiation response in DU145 cells. The surviving fraction at 2 Gy (SF2) of cells treated with CPA-7 and radiation was 0.52 compared to 0.74 in cells treated with radiation alone. Treatment of cells with 2 μM CPA-7 had little to no effect on the SF2.

### Example 5

### TUNEL Assay Demonstrates Increased Apoptosis of DU145 Cells

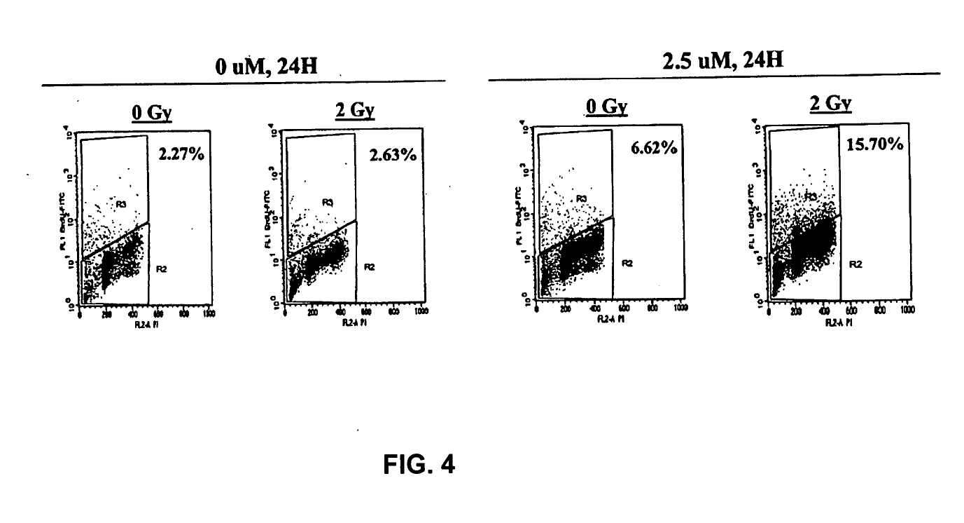

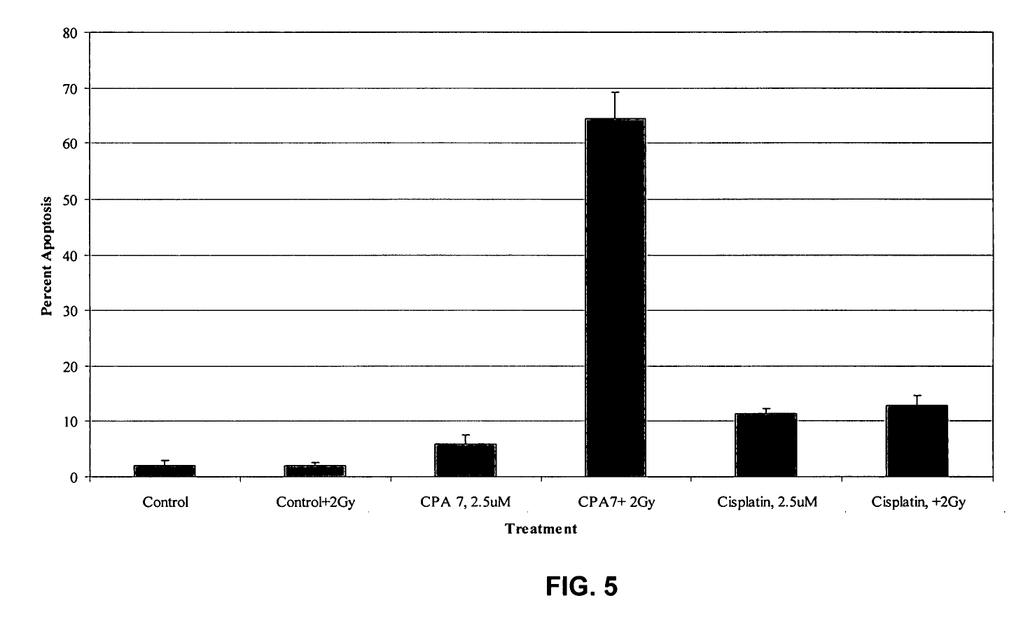

The enhancement of radiation responsiveness was also evident when apoptosis was measured using a TUNEL assay (FIGS. 4 and 5). DU145 cells were pre-treated for 4 hours with CPA-7 or vehicle alone prior to radiation. A TUNEL assay was performed 24 hours post-radiation and the results are depicted in FIG. 4. At 24 hours, only 2% of untreated cells had undergone spontaneous apoptosis. The baseline apoptotic rate was not increased significantly by irradiation with 2 Gy (2.63% of cells). CPA-7 treatment alone increased the baseline apoptotic rate to 6.62% at 24 hours. In contrast, 15.7% of cells treated with both radiation and CPA-7 had undergone apoptosis at 24 hours, indicative of a synergistic effect. FIG. 5 depicts the results of a similar assay in which cells were pre-treated with CPA-7 for 24 hours prior to irradiation. While there was no alteration in the background apoptotic rate in untreated cells, or those treated with either radiation or CPA-7 alone, the cells treated with CPA-7 for 24 hours prior to radiation had a apoptosis rate of over 60%. Additionally, not all platinum complexes have this effect. The apoptosis rate observed with Cisplatin in combination with radiation was not significantly enhanced. Other reports have demonstrated that Cisplatin does not modulate Stat3 activation.

### Example 6

### Effect of Platinum Complex and Radiation on Cell Viability

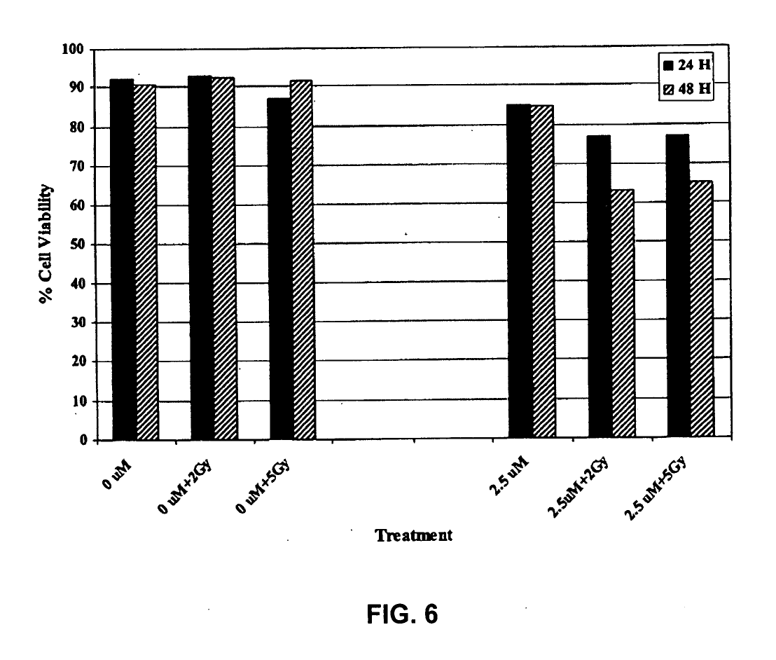

Cell viability after treatment with CPA-7 followed by radiation was also measured. FIG. 6 shows the effect of CPA-7, radiation and combined treatment (CPA-7+radiation) on cellular viability as measured by a trypan blue exclusion assay. The cells were pre-treated for 4 hours with CPA-7 or vehicle alone and then irradiated or mock irradiated. The trypan blue exclusion assay was completed at 24 and 48 hours after radiation. As expected, no difference in baseline cellular viability was observed after treatment with 2 Gy of radiation (91% viability) when compared to untreated controls (90%) after 48 hours of incubation. CPA-7 treatment alone did induce a small decrease in cellular viability as measured by trypan blue exclusion. At 48 hours, 85% of cells treated with CPA-7 were viable compared with 90% of cells in untreated controls. In contrast, only 63% of cells treated with CPA-7 and 2 Gy of radiation were viable at the 48-hour time point, once again indicative of a synergistic effect of the combination of CPA-7 and radiation.

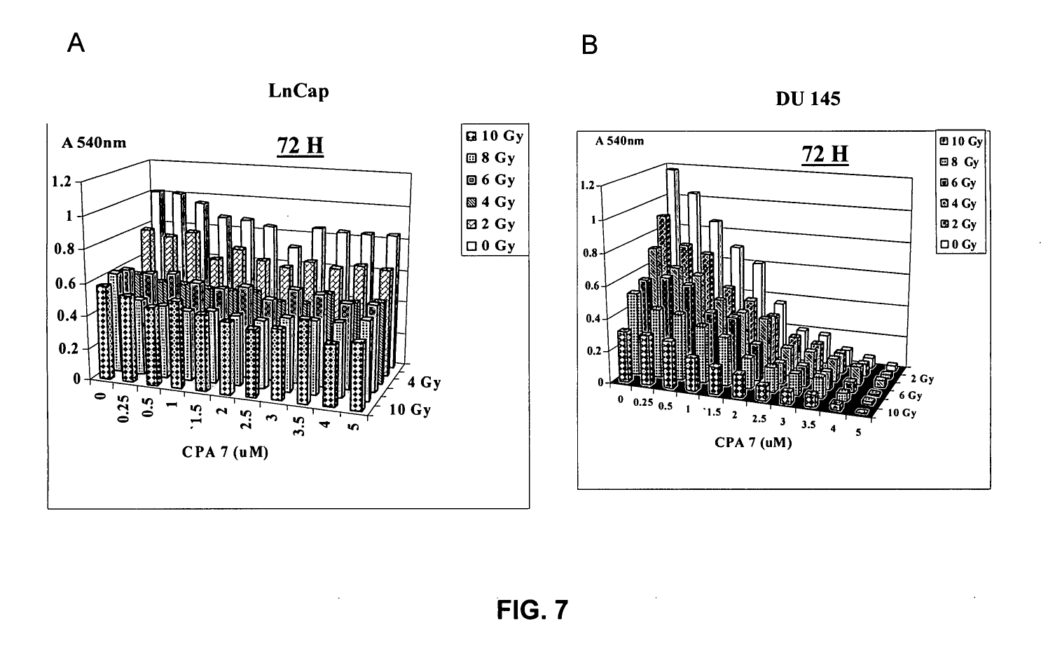

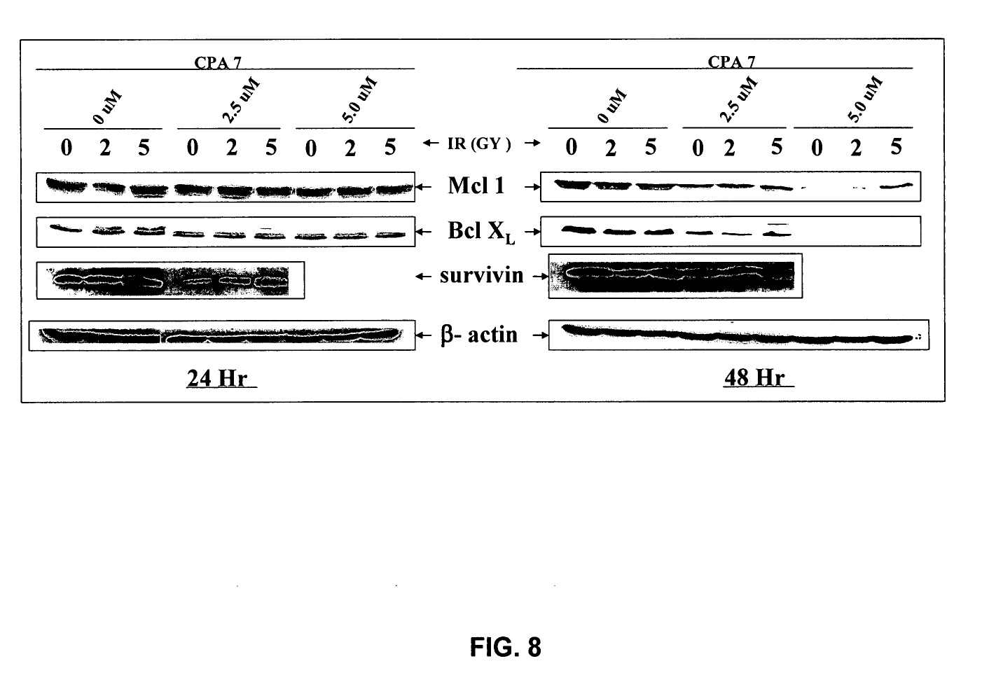

FIG. 8 depicts the results of an MTT assay for cellular viability and compares LnCap cells that do not have activated Stat3 (FIG. 7A) to DU145 cells which do have activated Stat3 (FIG. 7B) after treatment with various amounts and combinations of CPA-7 and radiation. The cells were pretreated for 24 hours with the indicated amount of CPA-7 or vehicle alone and then radiated with the indicated dose of Gys. The MTT assay was completed 72 hours post-radiation. As shown in FIG. 7A, the CPA-7 did not enhance the radiation response of the LnCap cells which do not express activated Stat3. In contrast, as shown in FIG. 7B, treatment with even a small amount of CPA-7 enhanced the radiation responsiveness of the DU145 cells.

### Example 7

### Survivin and Other Stat3 Regulated Proteins are Down-Regulated by CPA-7 Treatment

Previous studies demonstrated that Stat3 directly binds and regulates the survivin promoter and results in down-modulation of several proteins involved in blocking apoptosis of cells. To determine whether CPA-7 was affecting the expression of any of these proteins, DU145 cells were pretreated for four hours with CPA-7 or vehicle alone and irradiated. The cells were harvested either 24 hours or 48 hours later for Western blot analysis. As shown in FIG. 8, treatment with CPA-7 down-regulated survivin expression after 24 hours of incubation, suggesting that this might be the mechanism by which CPA-7 is inducing its radiation response enhancement effect. In contrast, radiation alone did not alter the overall levels of survivin. Interestingly, survivin expression levels returned to baseline at 48 hours. Furthermore, other downstream effectors in the Stat3 pathway were also affected by CPA-7. Bcl-X_{L} and Mcl 1, two anti-apoptotic proteins, were both down-regulated in CPA-7 treated cells after 48 hours of incubation, again suggestive that CPA-7 is acting by inhibiting the Stat3 pathway.

We claim:

1. A method of enhancing the radiation response of a cell having activated Stat 1, Stat3, or Stat5 comprising contacting the cell with an effective amount of a platinum complex selected from the group consisting of CPA-1, CPA-3, CPA-7 and pharmaceutically acceptable salts thereof and contacting the cell with an effective amount of radiation, wherein the radiation response of the cell is enhanced.

2. The method of claim 1, wherein the cell is a cancer cell, a tumor cell or a transformed cell.

3. The method of claim 2, wherein the cancer cell is selected from the group consisting of a breast cancer cell, a lung cancer cell, an ovarian cancer cell, a head and neck cancer cell, a melanoma cell, a prostate cancer cell, a multiple myeloma cell, a lymphoma cell, a leukemia cell, a gastric cancer cell, a glioma cancer cell, an ovary cancer cell, a colon cancer cell, and a pancreatic cancer cell.

4. The method of claim 1, wherein the cell is a human cell.

5. The method of claim 1, wherein the cell has activated Stat3.

6. The method of claim 1, wherein the platinum complex is CPA-7.

7. The method of claim 1, wherein the cell is contacted with the platinum complex prior to contacting the cell with the radiation.

8. The method of claim 1, wherein the cell is contacted with the platinum complex from about 1 hour to about 2 days prior to contacting the cell with the radiation.

9. A method of affecting a cell having activated Stat1, Stat3, or Stat5 comprising contacting the cell with a synergistically effective combination of radiation and CPA-7 or a pharmaceutically acceptable salt thereof.

10. The method of claim 9, wherein the cell is a cancer cell, a tumor cell or a transformed cell.

11. The method of claim 9, wherein the cell is a human cell.

12. The method of claim 9, wherein the cell has activated Stat3.

13. The method of claim 9, wherein the radiation is selected from the group consisting of X-rays, γ-rays, radioactive seeds and radionuclides.

14. The method of claim 9, wherein the cell is contacted with the CPA-7 prior to contacting the cell with the radiation.

15. The method of claim 10, wherein the cell is contacted with the CPA-7 from about 1 hour to about 2 days prior to contacting the cell with the radiation.

16. A method of affecting a cell in a subject, the cell having activated Stat1, Stat3, or Stat5, comprising administering to the subject a synergistically effective combination of radiation and CPA-7 or a pharmaceutically acceptable salt thereof.

17. The method of claim 16, wherein the subject is a human.

18. The method of claim 16, wherein the cell is a cancer cell, a tumor cell or a transformed cell.

19. The method of claim 16, wherein the cell has activated Stat3.

20. The method of claim 16, wherein the cell is contacted with the CPA-7 prior to contacting the cell with the radiation.

21. The method of claim 16, wherein the cell is contacted with the CPA-7 from about 1 hour to about 2 days prior to contacting the cell with the radiation.

22. The method of claim 16, wherein the administration of the CPA-7 lowers the amount of radiation required for cellular effects.

23. A method of enhancing the radiation response of a cell in a subject, the cell having activated Stat1, Stat3, or Stat5, comprising administering to the subject an effective amount of a platinum complex selected from the group consisting of CPA-1, CPA-3, CPA-7 and pharmaceutically acceptable salts thereof and administering an effective amount of radiation, wherein the radiation response of the cell is enhanced.

24. The method of claim 23, wherein the subject is a human.

25. The method of claim 23, wherein the cell is a cancer cell, a tumor cell or a transformed cell.

26. The method of claim 23, wherein the cell has activated Stat3.

27. The method of claim 23, wherein the platinum complex is CPA-7.

28. The method of claim 23, wherein the cell is contacted with the platinum complex prior to contacting the cell with radiation.

29. The method of claim 23, wherein the cell is contacted with the platinum complex from about 1 hour to about 2 days prior to contacting the cell with the radiation.

30. The method of claim 23, wherein the radiation is selected from the group consisting of X-rays, γ-rays, radioactive seeds and radionuclides.

31. The method of claim 23, wherein the administration of the platinum complex lowers the amount of radiation required for cellular effects.
